(19) United States
(12) Patent Application Publication
(10) Pub. No.: US 2005/0019803 A1
(43) Pub. Date: Jan. 27, 2005

(54) Array electrode

(76) Inventors: Timothy Z. Liu, Fremont, CA (US); Timothy Woudenberg, Moss Beach, CA (US); Jer-Kang Chen, Palo Alto, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/866,640

(22) Filed: Jun. 12, 2004

Related U.S. Application Data
(60) Provisional application No. 60/478,319, filed on Jun. 13, 2003.

(51) Int. Cl. C12Q 1/68; C12M 1/34

(52) U.S. Cl. 435/6; 435/287.2; 427/2.11

(57) ABSTRACT

Disclosed herein is an array patterned on an electrode and a method for fabricating the same. The array comprises a probe covalently attached to the electrode through a coupling group, an attachment group, and an aryl group.

### CROSS-REFERENCE TO RELATED APPLICATION

This application claims the benefit of U.S. Provisional Patent Application No. 60/478,319, filed on Jun. 13, 2003, the disclosure of which is incorporated by reference.

### FIELD OF THE INVENTION

The present application relates generally to detecting of biomolecules, and, more particularly, to arrays and microarrays fabricated on diamond electrodes, which are useful for detecting biomolecules.

### INTRODUCTION

Direct and indirect electrochemical detection of biomolecules, for example, DNA or proteins, is an alternative to fluorescent detection. Electrochemical detection using biomolecule microarrays features low detection limits, portability, and reduced instrumentation costs. Microarrays using electrochemical detection of targets are fabricated upon a conductive substrate.

### SUMMARY OF THE INVENTION

An aspect of the present disclosure provides an array comprising a substrate, wherein the substrate is electrically conductive; and a plurality of array elements, wherein each array element comprises a probe covalently attached to the substrate through a coupling group, an attachment group, and an aryl group.

Another aspect provides a method for manufacturing an array comprising a substrate, wherein the substrate is electrically conductive; and a plurality of array elements, wherein each array element comprises a probe covalently attached to the substrate through a coupling group, an attachment group, and an aryl group. The method comprises at least the steps of: derivatizing the surface of the substrate by electrochemically reducing an aryl azide and thereby covalently attaching the aryl group to the substrate, wherein the aryl azide comprises an attachment group; and conjugating a probe to the attachment group through a coupling group.

In some embodiments substrate is conducting or semiconducting diamond, for example, boron-doped diamond. In some embodiments, the array comprises at least about 100 array elements. In some embodiments, the probe is a biomolecule, for example, DNA.

In some embodiments, the coupling group is selected from the group consisting of nitrogen, oxygen, and sulfur. In some embodiments, the attachment group is derived from a group selected from the group consisting of alkyl halides, activated esters, and acid chlorides. In some embodiments, the aryl group is 1,4-phenylene.

Some embodiments further comprise a resist applied to the substrate, wherein resist comprises openings through which the array elements are exposed.

### DETAILED DESCRIPTION OF THE PREFERRED EMBODIMENTS

An embodiment provided herein is an array or microarray fabricated on a conductive substrate or electrode. Arrays and microarrays are useful in the detection and identification of chemical compounds, and in particular, the identification of biomolecules. An array comprises a plurality of array elements patterned on a substrate. Typically, each array element has an “address,” which is, for example, its position on the substrate. Each array element comprises one or more probe compounds selected to bind to or react with a particular target compound or compounds. Because the address of each array element is known, a user can identify a target or targets in a sample by exposing the array to the sample and identifying to which array elements the target(s) are bound, which permits a user to monitor complex systems, for example, changes in gene expression over time, or the effects of a drug on a population. As used herein, the term “array” encompasses an array as well as a microarray.

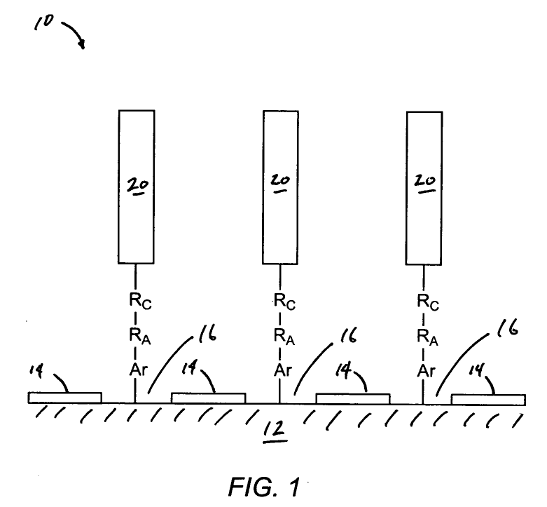

FIG. 1 illustrates a side view of an embodiment of an array 10 fabricated on an electrically conductive substrate 12, which is also referred to herein as an “electrode.” The illustrated array 10 comprises an optional resist applied to the surface of the electrode 14. The resist creates boundaries between the array elements 16. Other embodiments do not use a resist 14. Three array elements 16 are illustrated in FIG. 1. Each array element 16 comprises a probe 20 attached to the substrate 12 through an aryl group Ar, an attachment group R_{A}, and a coupling group R_{C}. Typically, the probe 20 is different for each array element 16.

The array 10 comprises at least two array elements 16 patterned on the substrate 12, which, in the present embodiment, is an electrode. An array element or element is an addressable unit of the array. The number of elements in an array constructed according to the present disclosure may be at least about 10, at least about 100, at least about 1000, at least about 10,000, at least about 100,000, at least about 10⁶, at least about 10⁷, at least about 10⁸, or at least about 10⁹. Some examples of arrays constructed according to the present disclosure have from about 10 to about 10⁹ elements. Other examples have from about 100 to about 10⁸ elements, from about 1,000 to about 10⁷ elements, and from about 10,000 to about 10⁶ array elements.

In some embodiments, the spacing of the array elements may is uniform. In other embodiments, the spacing varies. As will be apparent to one skilled in the art, a closer interelement spacing permits the construction of a physically smaller array. In some embodiments, the center-to-center element spacing is on the order of about 100 μm, about 10 μm, about 1 μm, or smaller. By way of example, in some embodiments, an array of over 10,000 elements is fabricated on a substrate with the dimensions of a standard microscope slide. A 100 μm center-to-center spacing of the array elements provides an array of over 100,000 elements in a standard 96-well microtiter plate. In some embodiments, the density of the array elements is at least about 1/cm², at least about 100/cm², at least about 10,000/cm², at least about 10⁶/cm², or at least about 10⁸/cm².

In some embodiments, the pattern of the array elements is a square grid, a rectangular grid, a hexagonal grid, or any pattern or combination of patterns desired. In other embodiments, the array elements are laid-down randomly, then interrogated to determine their addresses. Any geometric arrangement of array elements may be used, so long as the elements are addressable. The size of the elements depends on the particular application. For example, smaller array elements are advantageously used to detect smaller amounts of a target, for example, DNA. Those skilled in the art will understand that microarrays typically require smaller elements. In some embodiments, a single array comprises elements of different sizes. In some embodiments, the array elements are patterned by contact printing, which deposits as little as a few nanoliters of a solution of the probe onto the substrate. Other embodiments use ink-jet printing to pattern the array elements. One of ordinary skill will appreciate that the size of an element is limited, in part, by the interelement spacing.

In some embodiments, the surface of the substrate 12 is substantially smooth. In other embodiments, the substrate is not smooth. For example, in some embodiments, the array elements are applied in depressions or on raised features of the substrate. In some embodiments, a plurality of array elements is patterned on each depressed or raised feature. In the example cited above in which an array with 10,000 elements was formed on a standard 96-well plate, each well contains about 100 elements. In some embodiments, the depressions or raised areas are adapted to provide a particular environment for the array elements patterned therein or thereon, for example, by retaining a solution or to facilitate drying.

In some embodiments, the array 10 further comprises indicia or markings to provide or assist in addressing or locating the array elements. In some embodiments, these indicia are used in patterning the array, for example, as reference point for a printing device. In some embodiments, the depressed and/or raised features described above also serve these purposes.

The substrate 12 is any suitable electrode material that is covalently derivatized by electrochemical reduction of an aryl diazonium salt, which is described in greater detail below. Suitable substrates include conductive diamond, carbonaceous materials, iron, steel, silicon, and germanium. Carbonaceous electrodes include, for example, glassy carbon, carbon nanotubes, and highly-ordered/oriented pyrolytic graphite (HOPG).

In its pure state, diamond is an insulator, with a band gap of 5.5 eV, but may be made semiconducting or conducting by doping, for example with boron, lithium, nitrogen, phosphorus, sulfur, chlorine, arsenic, or selenium using methods known in the art. For example, samples of chemical vapor deposition (CVD) deposited boron-doped diamond films have resistivities of less than 0.1 Ω·cm. Methods for the chemical vapor deposition of boron-doped diamond films are known in the art and include filament assisted CVD and plasma enhanced CVD. Methods for fabricating boron-doped diamond electrodes are disclosed, for example, in U.S. Pat. Nos. 6,267,866, 5,900,127, 5,776,323, and 5,399,247, the disclosures of which are incorporated by reference.

In some embodiments, the substrate 12 is a conductive diamond electrode. As used herein, an electrode fabricated from conducting or semiconducting diamond is referred to as a “diamond electrode.” Conductive diamond has a large potential window in aqueous solutions, which permits the detection of species that react at high potentials. As used herein, the “potential window” of an electrode material in a particular medium is the range of potentials under which electrochemical reactions of the medium that interfere with desired electrochemical reactions do not occur. In some embodiments, the potential window of a diamond electrode in an aqueous medium is at least about 1.5 V or at least about 2 V. Some embodiments of diamond electrodes also display low background current, providing improved sensitivity. Other advantages of diamond as an electrode material include long-term stability, which is a consequence of the chemical inertness of diamond, and relative insensitivity to dissolved oxygen. Furthermore, the transparency of diamond facilitates optical methods in the fabrication and/or use of an array fabricated thereon. For example, the transparency permits optically detecting binding using a single detector on array patterned on both sides of the substrate. For example, an ECL signal is detectable through a diamond electrode as described in U.S. patent application Ser. No. 10/713,479, filed Nov. 14, 2003, the disclosure of which is incorporated by reference. For these reasons, arrays made with conductive diamond electrodes have significant advantages compared to arrays made from other electrode materials such as platinum, gold, and carbon.

The probe 20 is any probe useful in detecting a target compound. Microarrays are advantageously used to detect biomolecule targets or analytes, for example nucleic acids, proteins, polysaccharides, small molecules, and combinations thereof. In such microarrays, each array element 16 comprises one or more probes 20 that bind to a target biomolecule. Suitable biomolecular probes include DNA, RNA, PNA, proteins, polypeptides, polysaccharides, and the like.

The aryl group Ar, attachment group R_{A}, and coupling group R_{C} are discussed in greater detail below.

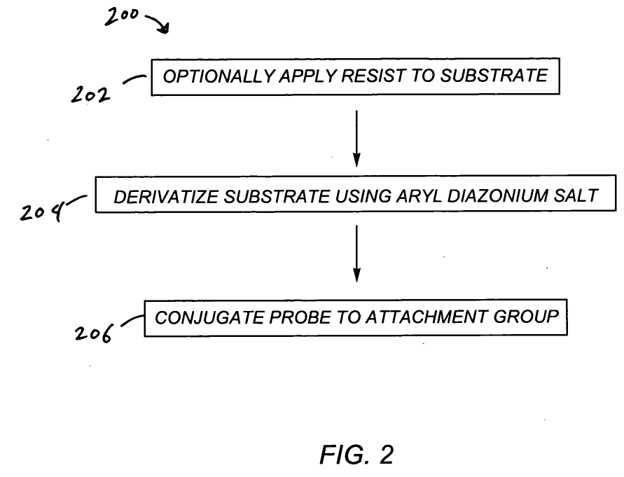

A method 200 for fabricating the disclosed array is provided in FIG. 2 with reference to FIG. 1. In step 202, a resist 14 is optionally applied to the surface of the electrode 12. In step 204, the electrode 12 is derivatized with an aryl diazonium salt bearing an attachment group R_{A}, as discussed in greater detail below. In step 206, a probe 20 is conjugated to the attachment group R_{A} through a coupling group R_{C}, as discussed in greater detail below. An advantage of a multistep process for derivatizing the electrode 12 followed by conjugating the probe 20 is that the reaction conditions for the respective steps may be optimized compared to a single-step method. For example, maintaining the biological function of a biomolecular probe 20 is more likely when coupled to the attachment group R_{A} in aqueous solution under mild conditions in step 206. Derivatizing the electrode 12 in step 204, on the other hand, is typically performed in an organic solvent.

In step 202, the electrode 12 is optionally coated with a resist 14. In some embodiments, the resist limits the spread of the array elements 16 applied in step 204. Examples of suitable resist materials include polylysine, aminosilanes, or amino-reactive silanes. In other embodiments, the resist 14 is a photoresist. In these embodiments, the surface of the substrate 12 is coated with a photoresist, which is then photolithographically patterned and etched to expose the electrode 12 through the photoresist, thereby permitting the user to control the spot or array element size. In some embodiments, the opening in the resist ranges from as small as 50 μm to as large as desired. The size of the opening in the photoresist limits the spot size because the disclosed method detects targets bound to probes covalently attached to the electrode 12, but does not appreciably detect target binding to probes 20 attached to the resist 14. In some embodiments, using a resist 14 facilitates the fabrication of high-density arrays because the resist inhibits spreading of the printed spots. Arrays as disclosed herein in which the openings in the resist are relatively small are also known as microelectrode arrays. Some embodiments of microelectrode arrays provide superior signal-to-noise ratios for the detection of the analyte, as would be apparent to those skilled in the art. In some embodiments, the resist 14 is stripped from the array 10 after step 206.

In some embodiments, the resist is applied and patterned before the electrode is derivatized by the aryl diazonium salt in step 204. In another embodiment, the resist is applied and patterned after the derivatization by the aryl diazonium salt in step 204, and before the coupling to the probe 20 in step 206. In some of these embodiments, the entire surface of the electrode 12 is derivatized using an aryl diazonium salt and the resist 14 is applied over some of the derivatized areas. In other embodiments, the surface of the electrode 12 is derivatized only at the array elements.

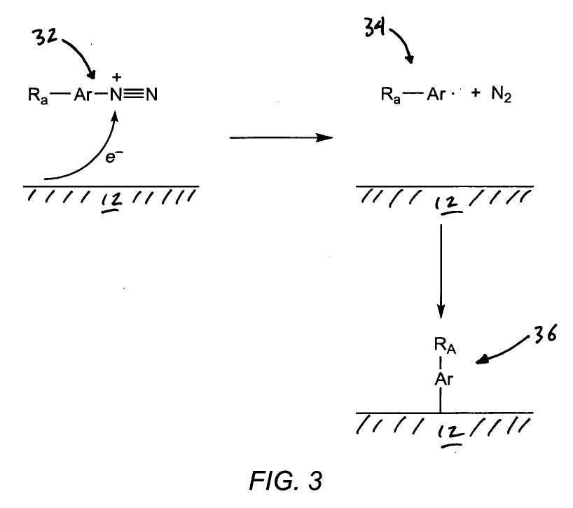

In step 204, the substrate 12 is derivatized with using an aryl diazonium salt. FIG. 3 illustrates the sequence of chemical reactions believed to occur in step 204. An aryl diazonium salt 32 is electrochemically reduced on or near the surface of the electrode 12, for example, by applying a reducing potential to the electrode 12. The aryl diazonium salt 32 comprises an aryl group Ar and an attachment group R_{A}. The reduction produces an aryl radical 34 that reacts with the electrode 12 forming an aryl species 36 covalently bound to the surface of the electrode 12. The derivatization reaction is generally performed in an organic solvent, for example acetonitrile. The potential for reducing the diazonium salt 32 is typically about −3 V vs. SCE in acetonitrile for a glassy carbon electrode. The surface coverage depends on a variety of factors including the concentration of the diazonium salt and the duration of the reaction. It will be appreciated that the derivatization reaction conditions may be varied to achieve particular results for different configurations of electrode materials and aryl diazonium salts. In some embodiments, the derivatization is performed in a series of steps, for example, for a subset of array elements in each step. In other embodiments, the derivatization is performed for all of the array elements in a single step.

The aryl group is any aryl group compatible with the derivatization reaction in step 204, including carbocyclic, heterocyclic, isolated, and fused aryl groups. Examples of suitable aryl groups include those based on benzene, naphthalene, anthracene, phenanthrene, biphenyl, pyridine, pyridazine, pyrimidine, and pyrazine. The geometric relationship between the covalent attachment of the aryl group Ar to the substrate 12 and the attachment group R_{A} permits the subsequent conjugation of the probe 20 to the attachment group R_{A} in step 206, as will be apparent to those skilled in the art. For example, in some embodiments, the relationship between the attachment to the substrate 12 and the attachment group R_{A} is 1,4 or 1,3, for example in aryl groups comprising six-membered rings. For fused systems such as naphthalene aryl groups, suitable relationships between the attachment to the substrate 12 and the attachment group R_{A} include 1,3-, 1,4-, 1,5-, 1,6-, 2,5-, 2,6-, 2,7-, and 2,8-relationships. Accordingly, in some embodiments, after the derivatization of the substrate 12 in step 204, the attachment group R_{A} is oriented away from the surface of the electrode 12. In some embodiments, the aryl group is substituted by one or more groups in addition to the attachment group R_{A}. These substituents are selected from the group consisting of alkyl, aryl, aralkyl, ether, amine, nitrile, halo, ester, amide, ketone, thiol, nitro, and combinations thereof.

The method illustrated in FIG. 3 is applicable to a wide variety of electrode materials. For example, diamond, carbon, iron, and steel electrodes are derivatized using this method, as described in Kuo et al. “Electrochemical Modification of Boron-Doped Chemical Vapor Deposited Diamond Surfaces with Covalent Bonded Monolayers” Electrochem. Solid-State Lett. 1999, 2:6, 288-290; Allongue et al. “Covalent Modification of Carbon Surfaces by Aryl Radicals Generated from the Electrochemical Reduction of Diazonium Salts” J. Am. Chem. Soc. 1997, 119, 210-207; and Adenier et al. “Covalent Modification of Iron Surfaces by Electrochemical Reduction of Aryldiazonium Salts” J. Am. Chem. Soc. 2001, 123, 4541-4549, the disclosures of which are incorporated by reference.

In step 206, the probe 20 is coupled to the attachment group R_{A} through a coupling group R_{C}. In some embodiments, the coupling group R_{C} is a group present in the native probe, for example, a thiol, alcohol, or amine group of a polypeptide, nucleic acid, or polysaccharide. In another embodiment, the probe is modified to comprise the coupling group R_{C}.

In some embodiments, probe 20 is coupled directly to the attachment group R_{A} in step 206. In other embodiments, attachment group R_{A} is converted into a modified attachment group R_{A′} after derivatization of the electrode 12 in step 204, but prior to coupling with the probe 20 in step 206. For example, in one embodiment, R_{A} is an amide that is deprotected to provide an amine R_{A′} group. In some embodiments, the modification involves several steps. For example, in other embodiments, R_{A} is a carboxylic ester, which is first converted into a carboxylic acid, then into an acid chloride R_{A′} group. In other embodiments, R_{A} is a nitro group, which is reduced to an amine R_{A′}. Appropriate R_{A} and R_{A′} groups are selected according to the particular application, as understood by those skilled in the art. In the discussion of the second, coupling reaction, it should be understood that the terms “attachment group” and “R_{A}” include modified attachment groups, R_{A′}.

As used herein, the terms “attachment group” and “R_{A}” are used to refer to the attachment group both before and after the reaction with the coupling group. Similarly, the terms “coupling group” and “R_{C}” refer to the coupling group both before and after the reaction with the attachment group.

Those skilled in the art will appreciated that a wide variety of attachment groups R_{A} and coupling groups R_{C} are useful in accordance with the disclosed invention, and that an attachment group in one embodiment may be a coupling group in another, and vice versa. The skilled artisan will also appreciate that certain attachment groups are especially compatible with certain coupling groups. For example, as described above, alkyl halides are compatible with amines, thiols, and alcohols. Carboxylic acids, acid halides, and esters are compatible with amines, thiols, and alcohols. The attachment and coupling groups may be an organic azide and an alkyne or nitrile, for example, the reactions of which provide triazoles or tetrazoles, respectively. In another embodiment, the attachment and coupling groups together form a metal-ligand coordination complex. In yet another embodiment, the attachment group is multifunctional, reacting with a plurality of coupling groups, increasing the concentration of the probe on the electrode, and consequently, improving the sensitivity of the assay.

In some embodiments, the attachment group R_{A} is an alkyl halide, for example, a chloride, bromide, or iodide, or an equivalent leaving group, for example, sulfonate ester, sulfimide, carboxylate ester, and the like. In other embodiments, the attachment group is an activated ester or equivalent, for example, a carboxylic ester, activated amides, hydroxamate, N-hydroxysuccinimide ester, acyl halide, and the like. In other embodiments, the attachment group R_{A} is a leaving group suitable for nucleophilic aromatic substitution, for example, fluoride or nitro.

In step 206, the probe 20 and coupling group R_{C} is contacted with the attachment group R_{A} using any means known in the art, for example, by contact printing or by inkjet printing. In other embodiments, an automated fluid handling device is used. The reactions for all of the array elements 16 are performed simultaneously in some embodiments. In other embodiments, the reactions are not all performed simultaneously.

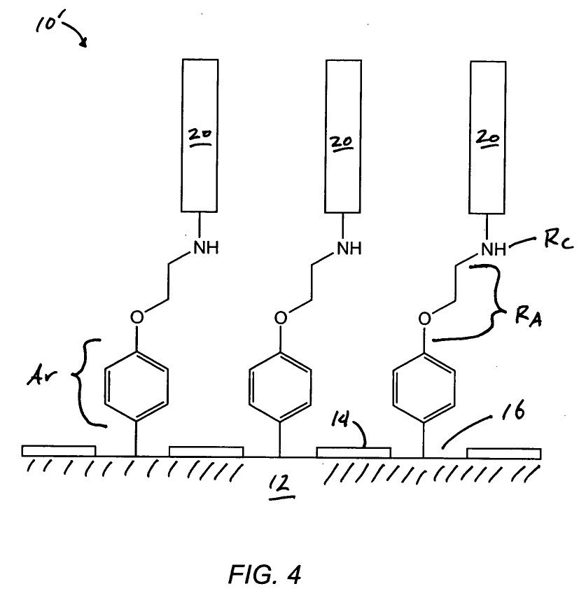

FIG. 4 illustrates an embodiment of the array 10′ fabricated on a conductive diamond substrate 12 in which R_{A} is —OCH₂CH₂Br, an alkyl bromide, and R_{C} is —NH₂ on an oligonucleotide probe 20. In the illustrated embodiment, the coupling reaction is performed in a single step at pH 8-8.5, in aqueous solution. This alkyl bromide coupling group is also useful for coupling to —NH₂ groups on exposed lysines of polypeptides. Other coupling groups compatible with an alkyl bromide attachment group include —SH, —OH, and the like.

The embodiments illustrated and described above are provided as examples only. Various changes and modifications can be made to the embodiments presented herein by those skilled in the art without departure from the spirit and scope of the teachings herein.

1. An array comprising:
a substrate, wherein the substrate comprises conducting or semiconducting diamond, and
a plurality of array elements, wherein each array element comprises a probe covalently attached to the substrate through a coupling group, an attachment group, and an aryl group.

2. The array of claim 1, wherein the conducting or semiconducting diamond comprises boron-doped diamond.

3. The array of claim 1, wherein the array comprises at least about 100 array elements.

4. The array of claim 1, wherein the probe is a biomolecule.

5. The array of claim 4, wherein the probe is DNA.

6. The array of claim 1, wherein the coupling group is selected from the group consisting of nitrogen, oxygen, and sulfur.

7. The array of claim 1, wherein the attachment group is derived from a group selected from the group consisting of alkyl halides, activated esters, and acid chlorides.

8. The array of claim 1, wherein the aryl group is 1,4-phenylene.

9. The array of claim 1, further comprising a resist applied to the substrate, wherein resist comprises openings through which the array elements are exposed.

10. A method for manufacturing an array, wherein
the array comprises a substrate, wherein the substrate wherein the substrate comprises conducting or semiconducting diamond, and
a plurality of array elements, wherein each array element comprises a probe covalently attached to the substrate through a coupling group, an attachment group, and an aryl group
the method comprising:
derivatizing the surface of the substrate by electrochemically reducing an aryl azide and thereby covalently attaching the aryl group to the substrate, wherein the aryl azide comprises an attachment group; and
conjugating a probe to the attachment group through a coupling group.

11. The method of claim 10, wherein the conducting or semiconducting diamond comprises is boron-doped diamond.

12. The method of claim 10, wherein the method comprises at least about 100 array elements.

13. The method of claim 10, wherein the probe is a biomolecule.

14. The method of claim 4, wherein the probe is DNA.

15. The method of claim 10, wherein the coupling group is selected from the group consisting of nitrogen, oxygen, and sulfur.

16. The method of claim 10, wherein the attachment group is a group selected from the group consisting of alkyl halides, activated esters, and acid chlorides.

17. The method of claim 10, wherein the aryl group is 1,4-phenylene.

18. The method of claim 10, further comprising applying a resist to the substrate, wherein resist comprises openings through which the array elements are exposed.
